(19) United States
(12) Patent Application Publication
(10) Pub. No.: US 2015/0045620 A1
(43) Pub. Date: Feb. 12, 2015

(54) CURVED MANUAL SURGICAL EQUIPMENT

(71) Applicants: INDUSTRY- UNIVERSITY COOPERATION FOUNDATION HANYANG UNIVERSITY ERICA CAMPUS, Ansan-si Gyeonggi-do (KR); KOHYOUNG TECHNOLOGY INC., Seoul (KR)

(72) Inventor: YoungSik Kwon, Ansan-si (KR)

(21) Appl. No.: 14/005,032

(22) PCT Filed: Apr. 17, 2013
(86) PCT No.: PCT/KR2013/003249

§ 371 Date: Sep. 13, 2013

(30) Foreign Application Priority Data
Apr. 27, 2012 (KR) 10-2012-0044923

(51) Int. Cl. A61B 19/00; A61B 1/00; A61B 1/05

(52) U.S. Cl. CPC A61B 19/22; A61B 1/05; A61B 1/00052
USPC 600/109; 606/130

(57) ABSTRACT

Provided is a curved manual surgical equipment allowing a plurality of surgical equipments to access affected areas. The curved manual surgical equipment includes: a curved frame; and a manual surgical equipment which includes a handle allowing an operator to control a surgical equipment, an internal connector allowing the surgical equipment to perform the translation and rotation motions, and an end effector attached to a tip of the internal connector to operate on affected areas and is inserted into an inner space of the curved frame in plural, wherein the internal connector is configured to include a plurality of rotating parts which performs translation and rotation motions within the curved frame; and a link which transfers pressure, tension, and rotating force between the plurality of rotating parts.

### TECHNICAL FIELD

The present invention relates to a curved manual surgical equipment, and more particularly, to a curved manual surgical equipment to allow a plurality of manual surgical equipments to access curved affected areas.

### BACKGROUND ART

When a human body has affected areas, such as wound or cyst, a surgery for treating the affected areas is performed. When affected areas, such as a limb and a body protrude to the outside and thus are easily accessed by surgical equipments, the affected areas may be easily operated by scissors, forceps, a coagulator, and the like, but when affected areas are positioned in deep places in the body, in particular, are positioned at curved passages, such as a larynx, a vagina, and a large intestine, and thus a field of vision is limited and it is difficult for a surgical equipment to be accessed thereto, which makes it difficult to perform a surgery.

As described above, in order to allow a surgical equipment to access affected areas at the curved passage, in general, by using medical instruments, such as a suspension laryngoscopy, a surgical equipment enters the affected areas after a straight passage from the outside to the affected areas is secured, thereby treating the affected areas.

For example, when affected areas are positioned at an occipital region, as illustrated in FIG. 1, after a straight passage is secured by positioning a patient's head back, and then a medical instrument for surgery 1 enters the occipital region through an oral cavity to treat the affected areas. However, when the affected areas are operated by positioning a patient's head back, a body may be damaged due to a compression of a neck and it is difficult to secure a straight passage which is sufficient to allow the medical instrument for surgery to enter the occipital region, depending on patient's physical characteristics. In particular, in the case of a patient having a short neck, a narrow occipital region, an overweight, a short lower jaw, and a short tongue or a disk patient, a patient with weak teeth, or a denture patient, a male patient, and the like, there may be often the case in which it is difficult to secure a straight passage from the outside to the affected area of the occipital region.

Therefore, when an affected area is positioned at a curved path, it may be difficult to operate on the affected area by allowing the surgical equipment to access the affected area.

In general, since the curved passage in body is narrow, it may be difficult to perform a surgery by allowing a plurality of manual surgical equipments to enter the curved passage at one time.

### DISCLOSURE

### Technical Problem

An object of the present invention is to provide a curved manual surgical equipment capable of performing a surgery by easily accessing curved affected areas.

Another object of the present invention is to provide a curved manual surgical equipment capable of allowing a plurality of manual surgical equipments to access affected areas at one time.

### Technical Solution

In one general aspect, there is provided a curved manual surgical equipment including: a curved frame; and a manual surgical equipment which includes a handle allowing an operator to control a surgical equipment, an internal connector allowing the surgical equipment to perform the translation and rotation motions, and an end effector attached to a tip of the internal connector to operate on affected areas and is inserted into an inner space of the curved frame in plural, wherein the internal connector is configured to include a plurality of rotating parts which performs translation and rotation motions within the curved frame; and a link which transfers pressure, tension, and rotating force between the plurality of rotating parts.

The rotating part coupled with a handle among the rotating parts 221 may be configured to include: a ball driving unit provided with a plurality of fixed projections; a high elastic rod elastically connecting the ball driving unit to one side of the rotating part; and a support part rotatably supporting the ball driving unit.

### Advantageous Effects

As set forth above, according to the curved manual surgical equipment according to the embodiments of the present invention, the accessibility of the surgical equipment to affected areas can be improved, thereby easily performing the surgery and reducing the inconvenience of a patient at the time of the surgery.

Further, according to the curved manual surgical equipment according to the embodiments of the present invention, the plurality of manual surgical equipments can be applied to affected areas at one time, thereby simplifying the surgery.

### DESCRIPTION OF DRAWINGS

The above and other objects, features and advantages of the present invention will become apparent from the following description of preferred embodiments given in conjunction with the accompanying drawings, in which:

FIG. 1 is a diagram illustrating an appearance in which after a straight passage is secured by positioning a patient's head back, a medical instrument for surgery according to the related art enters an occipital region through an oral cavity;

FIG. 2 is a perspective view of a curved manual surgical equipment according to an embodiment of the present invention;

FIG. 3 is a detailed diagram of the manual surgical equipment of FIG. 2;

FIG. 4 is a perspective view illustrating a coupled structure of a handle and a rotating part; and

FIG. 5 is a diagram illustrating a first control wire generating tension when the handle is bent.

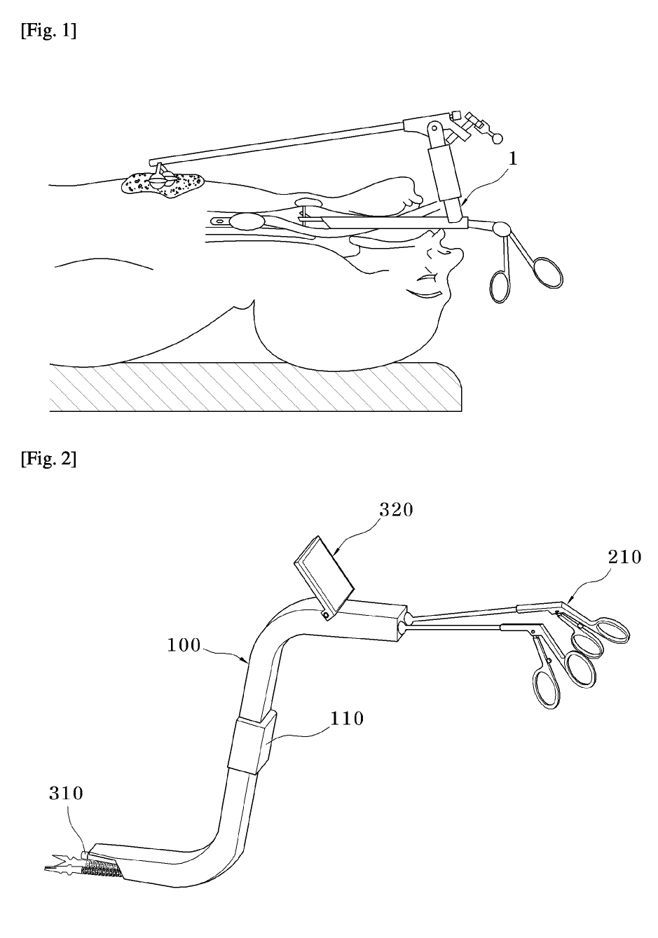

### BEST MODE

Hereinafter, a curved manual surgical equipment according to an embodiment of the present invention will be described in more detail with reference to the accompanying drawings.

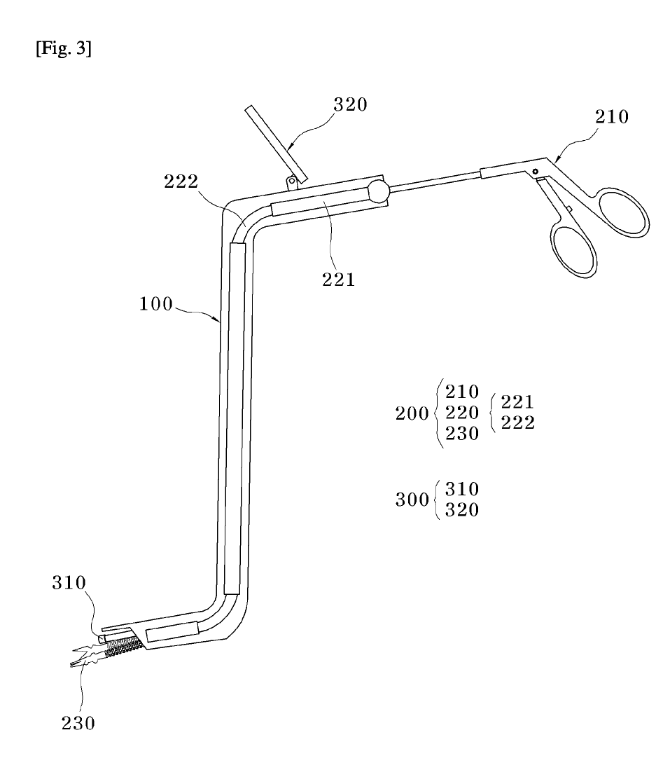

FIG. 2 is a perspective view of a curved manual surgical equipment according to an embodiment of the present invention and FIG. 3 is a detailed diagram of the manual surgical equipment of FIG. 2. The curved manual surgical equipment according to the embodiment of the present invention is configured to include a curved frame 100 bent to reach curved affected areas, such as an occipital region, and a plurality of manual surgical equipments 200 inserted into the curved frame and may further include an endoscope 300 through which affected areas may be seen.

The manual surgical equipment 200 is configured to include a handle 210 which allows an operator to control a surgical equipment, an internal connector 220 which allows the manual surgical equipment 200 to perform translation and rotation motions, and an end-effector 230 which is attached to a tip of the internal connector to operate on affected areas and the curved frame 100 may be provided with a mounting terminal 110 coupled with a mounting table.

A method of operating a curved manual surgical equipment according to the embodiment of the present invention will be simply described with reference to the case in which the curved manual surgical equipment is used to operate on an occipital region, by way of example. First, it is possible to secure a passage to allow the curved manual surgical equipment to reach an occipital region by naturally opening a patient's mouth in the state in which he/she lays down on the operating table looking at the ceiling. The operator inserts the curved frame 100 from which the end effector 230 of the curved manual surgical equipment according to the embodiment of the present invention into an oral cavity to reach the end effector 230 in the vicinity of the affected areas of the occipital region and mounts the curved frame 100 on the external mounting table. Next, the operator grips the handles 210 of the plurality of manual surgical equipments one by one in his/her both hands to rotate and translate the surgical equipments so as to allow the end effector 230 to be disposed at a position and a direction appropriate for the surgery of the affected areas, and then moves the handles 210 like scissoring to perform the operations, such as gripping, cutting, and coagulating the affected areas.

Since the curved manual surgical equipment according to the embodiment of the present invention moves the end effector 230 to a position and a direction appropriate for a surgery, the curved manual surgical equipment needs to perform the translation and rotation motions, which is achieved by allowing the internal connector 220 having the end effector 230 mounted at an end thereof to perform the translation and rotation motions within the curved frame 100.

The internal connector 220 is configured to include a plurality of rotating parts 221 which is able to perform the translation and rotation motions within the curved frame 100 and a link 222 which transfers pressure, tension, and rotating force between the plurality of rotating parts, in which the link serves to connect between the rotating parts by being smoothly bent at a bent portion of the curved frame. An example of the link may include a spring guide having a high elastic rod, such as a high elastic carbon tube, disposed at a center thereof, in which the spring guide may transfer a force pushing the internal connector to the rotating part and the high elastic rod may transfer a force pulling the internal connector or a force rotating the internal connector to the rotating part. For reference, FIG. 3 illustrates the case in which the internal connector 220 is configured to include three rotating parts 221 and two links 222 connecting the three rotating parts.

Meanwhile, the rotating part coupled with the handle among the rotating parts further includes a component for controlling the end effector.

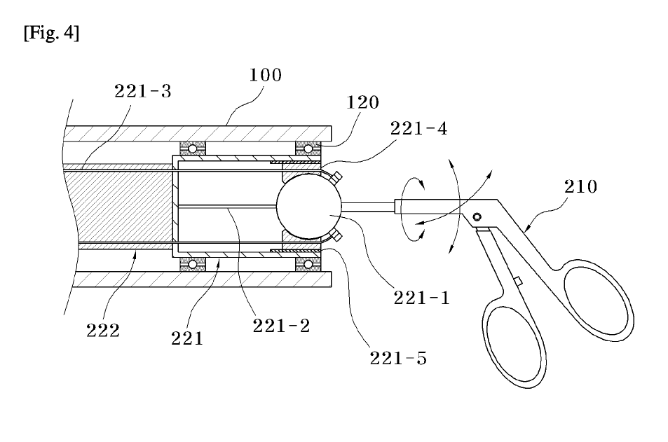

FIG. 4 is a perspective view illustrating a coupled structure of the handle and the rotating part. The rotating part coupled with the handle 210 is configured to include a ball driving part 221-1 formed with a plurality of fixed projections, a high elastic road 221-2 elastically connecting the ball driving part to one side of the rotating part, and a support part 221-4 rotatably supporting the ball driving part. The fixed projection of the ball driving part 221-1 is connected to a plurality of first control wires 221-3 of which the distal ends are connected the end effector and the first control wire 221-3 may maintain its position within the curved frame 100 by penetrating through a hole, a ring, and a guide (not illustrated), and the like, which are formed in the link 222.

An inner circumferential surface of the rotating part is provided with a slide groove 221-5 to allow the support part to be slid in a longitudinal direction of the rotating part and between the rotating part 221 and an inner circumferential surface of the curved frame 100 may be provided a bearing 120 to allow the rotating part 221 to be smoothly rotated.

According to the embodiment of the present invention, the translation and rotation motions of the end effector are controlled by the handle. In other words, a force is repeatedly transferred in such a manner that when the handle is pushed, pulled, or rotated, pressure, tension, or rotating force is transferred to the rotating part and the pressure, the tension, or the rotating force transferred to the rotating part is transferred a next rotating part by the link, such that the end effector may perform the translation and rotation motions. That is, the translation and rotation motions of the end effector may be easily controlled by the translation and rotation motions of the handle.

Meanwhile, the bending of the end effector is controlled by vertically and horizontally moving the handle and the control is able to be performed by allowing the plurality of first control wires 221-3 coupled with the ball driving part 221-2 to transfer tension to the end effector. As illustrated in FIG. 4, the ball driving part 221-1 is rotatably supported by the support part 221-4 and the ball driving part 221-1 is formed with the fixed projects at equidistance as many as the number of first control wires 221-3 so that the fixed projections may be coupled with one ends of the first control wires 221-3. The first control wires 221-3 of which the one ends are coupled with the fixed projections pass through the inside of the curved frame so as to be coupled the other ends thereof with the end effector. By the above configuration, when the handle moves vertically and horizontally, the ball driving part 221-1 rotates and the first control wire 221-3 applied to tension among the first control wires 221-3 of which the one ends are coupled with the fixed projections of the ball driving part 221-1 may pull the end effector to face the end effector in a desired direction.

As a result, the translation, rotation, bending motions are controlled depending on the motions of the handle 210.

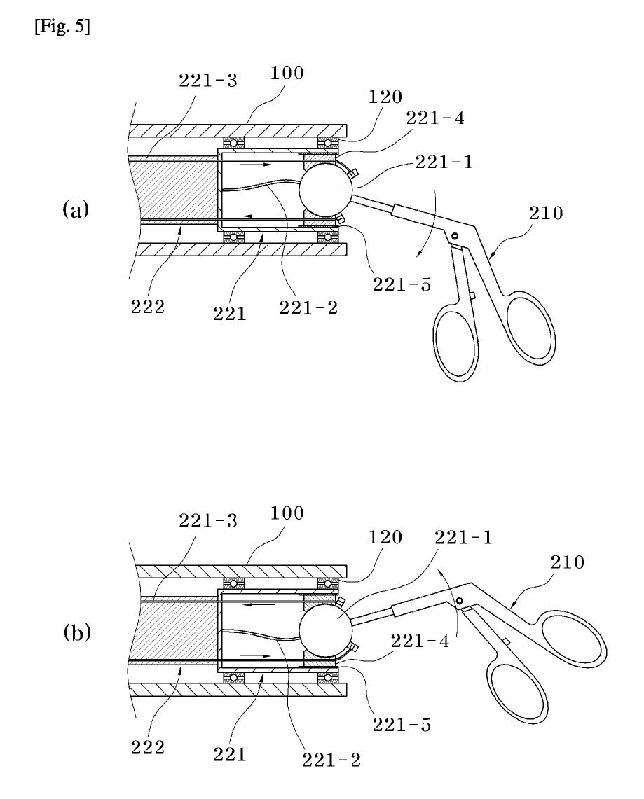

FIG. 5 illustrates the first control wire 221-3 generating tension when the handle is bent. When an operator bends the handle 210 clockwise (FIG. 5A), the ball driving part 221-1 rotates clockwise and the upper first control wire coupled with the fixed projection generates tension to bend the end effect toward the upper first control wire. To the contrary, when an operator bends the handle 210 counterclockwise (FIG. 5B), the lower first control wire generates tension to bend the end effector toward the lower first control wire. When the ball driving part 221-1 rotates by bending the handle 210, the ball driving part 221-1 is pulled to one side of the rotating part while the ball driving part 221-1 and the high elastic rod 221-2 connected to one side of the rotating part are bent. In this case, the handle part is pushed into the rotating part as a whole while the support part 221-4 supporting the ball driving part 221-1 is slid along the slide groove 221-5 formed on the inner circumferential surface of the rotating part.

Meanwhile, the curved manual surgical equipment according to the embodiment of the present invention moves by scissoring the handle to operate the end effector, such that the handle and the end effector are connected to each other by the second control wire (not illustrated). In this case, the second control wire has one end connected to the handle and penetrates through the rotating part and the link to have the other end thereof connected to the end effector. When the handle is unfolded and folded, the second control wire is tensioned to operate the end effector. In this case, a detailed method of driving the end effector may be various depending on a kind of end effector. For example, if it is assumed that the end effector moves by combining of two blades like forceps or scissors, the forceps or scissors of the end effector may be driven together by the scissoring of the handle and if it is assumed that the end effector performs an on-off operation like a coagulator or a suction pipe, the coagulator or the suction pipe may perform an on-off operation depending on the scissoring of the handle.

The link may be the spring guide having the high elastic rod embedded therein but any link transferring pressure, tension, and rotating force to translate and rotate the rotating part may be adopted. The internal connector may smoothly move at a bent portion in the curved frame.

Meanwhile, the internal connector may further include a locking wire to prevent the rotating part from perform a relative motion with respect to the curved frame. The locking wire is connected to a locking plate to press the rotating part to suppress the motion of the rotating part and only the locking wire may also press the rotating part.

The curved manual surgical equipment according to the embodiment of the present invention may further include an endoscope 300 configured to include an imaging lens 310 and a display device 320 displaying the captured image to see the affected areas and the imaging lens 310 may be provided at a portion at which the end effector 230 of the curved frame 100 protrudes and the captured image may be seen from the outside through the display device 320 provided at the outside of the curved frame 100.

Further, in the curved manual surgical equipment according to the embodiment of the present invention, the curved frame with the handle is separated in a Y-letter form and the manual surgical equipments are embedded in each of the separated curved frame, such that the operator may grip the handle at an appropriate interval.

### DETAILED DESCRIPTION OF MAIN ELEMENTS

100 CURVED FRAME
110 MOUNTING TERMINAL
120 BEARING
200 MANUAL SURGICAL EQUIPMENT
210 HANDLE
220 INTERNAL CONNECTOR
221 ROTATING PART
221-1 BALL DRIVING PART
221-2 HIGH ELASTIC ROD
221-3 FIRST CONTROL WIRE
221-4 SUPPORT PART
221-5 SLIDE GROOVE
230 END EFFECTOR
300 ENDOSCOPE
310 IMAGING LENS
320 DISPLAY DEVICE

1. A curved manual surgical equipment, comprising:
a curved frame 100; and
a manual surgical equipment 200 which includes a handle 210 allowing an operator to control a surgical equipment, an internal connector 220 allowing the surgical equipment to perform the translation and rotation motions, and an end effector 230 attached to a tip of the internal connector 220 to operate on affected areas and is inserted into an inner space of the curved frame 100 in plural,
wherein the internal connector 220 is configured to include a plurality of rotating parts 221 which performs translation and rotation motions within the curved frame 100; and a link 222 which transfers pressure, tension, and rotating force between the plurality of rotating parts 221.

2. The curved manual surgical equipment of claim 1, wherein the link 222 is a spring guide having a high elastic rod embedded therein.

3. The curved manual surgical equipment of claim 1, wherein a bearing 120 is further provided between the rotating part 221 and an inner circumferential surface of the curved frame 100.

4. The curved manual surgical equipment of claim 1, wherein the rotating part coupled with a handle among the rotating parts 221 is configured to include:
a ball driving unit 221-1 provided with a plurality of fixed projections;
a high elastic rod 221-2 elastically connecting the ball driving unit to one side of the rotating part; and
a support part 221-4 rotatably supporting the ball driving unit.

5. The curved manual surgical equipment of claim 4, wherein the plurality of fixed projections formed in the ball driving unit 221-1 are connected to the first control wires 221-3 of which the ends are connected to the end effector.

6. The curved manual surgical equipment of claim 4, wherein an inner circumferential surface of the rotating part 221 is formed with a slide groove 221-5 to allow the support part 221-4 to be slid in a longitudinal direction of the rotating part.

7. The curved manual surgical equipment of claim 1, wherein the curved frame 100 further includes a mounting terminal 110.

8. The curved manual surgical equipment of claim 1, further comprising:
an endoscope 300 configured to include an imaging lens 310 provided at a portion at which the end effector 230 of the curved frame 100 protrudes and a display device 320 displaying a captured image.

9. The curved manual surgical equipment of claim 1, wherein the curved frame 100 with the handle 210 is separated in a Y-letter form.
